(19) United States
(12) Patent Application Publication
(10) Pub. No.: US 2023/0138750 A1
(43) Pub. Date: May 4, 2023

(54) RESPIRATOR DEVICE AND METHOD

(71) Applicants: ISAAC STEINER, TEL AVIV (IL); ALEX GUREVICH, PETAH TIKVA (IL)

(72) Inventors: ISAAC STEINER, TEL AVIV (IL); ALEX GUREVICH, PETAH TIKVA (IL)

(21) Appl. No.: 17/911,899

(22) PCT Filed: Mar. 16, 2021
(86) PCT No.: PCT/IL2021/050292

§ 371 Date: Sep. 15, 2022

Related U.S. Application Data
(60) Provisional application No. 63/019,441, filed on May 4, 2020.
(60) Provisional application No. 62/991,653, filed on Mar. 19, 2020.

(51) Int. Cl. A61M 16/00; A62B 9/00

(52) U.S. Cl. CPC A61M 16/0003; A62B 9/006; A61M 2016/0015

(57) ABSTRACT

A device to protect a user from inhaling hazardous atmospheres including an insulated chamber having inlet and outlet. The chamber includes air heating means and air UV radiating means. The combined operation of the air heating means and the UV radiating means is configured for disinfecting the hazardous atmospheres. A cooling means is used for cooling the disinfected hazardous atmospheres. Wherein, the cooled disinfected hazardous atmosphere is then delivered to the user's breathing system.

### FIELD OF THE INVENTION

The present invention relates to device and method to protect a user from inhaling hazardous atmospheres in particular to protect a user from infectious viruses and diseases such as COVID-19.

### BACKGROUND OF THE INVENTION

A respirator is a device designed to protect the wearer from inhaling hazardous atmospheres, including particulate matter such as dust and airborne microorganisms such as but not limited to severe acute respiratory syndrome coronavirus 2 (SARS-CoV-2). Coronavirus disease 2019 (COVID-19) is an infectious disease caused by SARS-CoV-2. The respirator device is also designed to protect the wearer from inhaling hazardous atmospheres including also hazardous fumes, vapors and gases.

There are two main categories: the air-purifying respirator in which respirable air is obtained by filtering a contaminated atmosphere, and the air-supplied respirator in which an alternate supply of breathable air is delivered. Within each category, different techniques are employed to reduce or eliminate noxious airborne contaminants.

Air-purifying respirators range from relatively inexpensive single-use, disposable face masks sometimes referred to as a dust mask to more robust reusable models with replaceable cartridges often called a gas mask.

All respirators have some type of facepiece held to the wearer's head with straps, a cloth harness, or some other method. The facepiece of the respirator covers either the entire face or the bottom half of the face including the nose and mouth. Half-face respirators can only be worn in environments where the contaminants are not toxic to the eyes or facial area. For example, someone who is painting an object with spray paint could wear a half-face respirator, but someone who works with chlorine gas would have to wear a full-face respirator. Facepieces come in many different styles and sizes, to accommodate all types of face shapes. The differences in respirator design impact the respirator assigned protection factors, i.e. the resulting degree of protection for each kind of hazard.

Most types of respirators depend upon forming a good seal between the respirator body and the face of the wearer. Fit testing procedures have been developed to ensure that the respirator is appropriate for the wearer and the wearer's donning technique is capable of creating an adequate seal.

In the United States, the National Institute for Occupational Safety and Health defines categories of particulate filters for example: N95 Filters at least 95% of airborne particles. European standard EN 143 defines the classes of particle filters that can be attached to a face mask for example P3 Filters at least 99.95% of airborne particles. European standard EN 149 defines the classes of “filtering half masks” or “filtering face pieces” (FFP), that is respirators that are entirely or substantially constructed of filtering material, for example FFP3 filters at least 99% of airborne particles.

Another technique to stop airborne viruses' transmittal, instead of filtration, is killing the airborne viruses. When it comes to airborne disinfection of viruses, one of the generic methods to kill the virus is by heat.

Another generic method to kill viruses, including Covid-19, is by exposing the virus to UV radiation.
Simultaneous application of heating and UV exposure provides synergistic effect.

It was proved that simultaneous application has strong synergistic effect in viruses inactivation rate: it is about 10 time more efficient than in separate application of both disinfection technologies.

Certain materials (for example copper, platinum nano-colloid material, and silver nano-colloid material) have been proved to kill viruses coming in physical contact with said materials.

One object of the present invention is to provide a respirator device and method that can be reusable and thus, when a worldwide pandemic disease occurs there could be less lack of single-use, disposable face masks.

Another object of the present invention is to provide a respirator device and method that is reusable and less robust.

Another object of the present invention is to provide a system and method that can be used to disinfect hazardous air using the air conditioning system in vehicles, ships, air purifier, personal air conditioner, shelter air conditioning system, filtering systems for biological laboratories and buildings.

Another object of the present invention is to provide a respirator device that uses a bi-directional disinfection process to disinfect hazardous air both coming into the user's breathing system and coming out from an infected user wearing the device.

### SUMMARY OF THE INVENTION

The present invention relates to device and method to protect a wearer and its surroundings from a user inhaling and/or exhaling hazardous atmospheres in particular to protect a wearer and its surroundings from infectious diseases.

In accordance with an embodiment of the present invention there is provided a device to protect a user from inhaling hazardous atmospheres including an insulated chamber having inlet and outlet. The chamber includes air heating means and air UV radiating means. The combined operation of the air heating means and the UV radiating means is configured for disinfecting the hazardous atmospheres. A cooling means is used for cooling the disinfected hazardous atmospheres. Wherein, the cooled disinfected hazardous atmosphere is then delivered to the user's breathing system.

In accordance with an embodiment of the present invention there is provided a device to stop a user from exhaling hazardous atmospheres including an insulated chamber having inlet and outlet. The insulated chamber includes air heating means and air UV radiating means. The device further includes the use of surface elements such as said heating means coated with materials such as copper, platinum nano-colloid material, or other virus killing materials. The combined simultaneous operation of the air heating means, the UV radiating means, and the virus killing materials is configured for disinfecting the hazardous atmospheres. A cooling means is used for cooling the disinfected hazardous atmospheres. Wherein, cooling means can be placed in one or more locations for example but not limited to inside the insulated chamber, or attached to an air outlet valve thereby enabling the device to cool disinfected hazardous atmosphere both coming in to the user's breathing system or coming out of the user's breathing system.

In accordance with an embodiment of the present invention there is provided a device configured to perform a reverse operation wherein the system can operate in reverse direction: to eliminate viruses in air exhaled by an infected person.

In another aspect of the present invention there is provided a method for protecting a user from inhaling hazardous atmosphere comprising the steps of providing insulated chamber having an inlet, outlet. The insulated chamber insulates the hazardous atmosphere from a user. In the next step, the hazardous atmosphere is heated. In the next/simultaneous step, the hazardous atmosphere is exposed to UV radiation. In the next/simultaneous step, the hazardous atmosphere is exposed to virus killing materials such as copper, platinum nano-colloid material, or other virus killing materials. The step of heating and exposing the hazardous atmosphere to UV radiation and virus killing materials causes the hazardous atmosphere to be disinfected by killing any viruses. In the next step the disinfected air is cooled to a temperature suitable for the user's breathing system for example to the user current environment temperature. The cooled disinfected atmosphere is then delivered to the user's breathing system.

Surface elements of the present invention, such as but not limited to the heat exchanger element, are exposed to the air flow and can be coated by virus killing materials (for example copper, platinum nano-colloid material and silver nano-colloid material) thus enhancing the disinfection process coming in contact with the air inside the insulated chamber.

In another aspect of the present invention there is provided a method for bi-directional disinfection process of hazardous atmosphere comprising the steps of providing a filter, an insulated chamber coated with virus killing materials such as copper, platinum nano-colloid material, silver nano-colloid material, having an inlet, outlet. The incoming air goes through a filter, to the insulated chamber that insulates the hazardous atmosphere from a user. In the next step, the hazardous atmosphere is heated and Simultaneously or separately exposed to UV radiation. The step of heating and exposing the hazardous atmosphere to UV radiation in combination with the use of copper, platinum nano-colloid material or other virus killing material coated on the surface elements such as the heating element causes the hazardous atmosphere to be disinfected by killing any viruses. In the next step the disinfected air is cooled to a temperature suitable for the user's breathing system for example to the user current environment temperature. The cooled disinfected atmosphere is then delivered to the user's breathing system. Exhaled air goes to the insulated chamber that insulates the hazardous atmosphere from a user. In the next step, the hazardous atmosphere is heated and Simultaneously or separately exposed to UV radiation and to copper or platinum nano-colloid materials or other virus killing material which are coated on the surface elements such as the heating element. The step of heating and exposing the hazardous atmosphere to UV radiation in combination with the use of copper or platinum nano-colloid material or other virus killing material coated on the surface elements such as the heat exchanging element causes the hazardous atmosphere to be disinfected by killing any viruses. In the next step the disinfected air can be cooled for example to the user current environment temperature. The disinfected atmosphere is then released to the user's surroundings.

In another aspect of the present invention there is provided a method for a desktop device facing a user, delivering disinfected air to the user's facial area without having a need for the user to wear a face mask. The desktop device is protecting a user while said user is sitting for example and without limitation in a workplace, dining room, restaurant and in the plane during flight. The device of the invention treats air, kills viruses and diffuses clean disinfected air to the direction of user's head such way that a user inhales air free of viruses. One of the advantages of this aspect of the invention is that a mask is not required, and protection is provided by generating a disinfected air curtain preventing penetration of infected air to a user breathing zone.

### BRIEF DESCRIPTION OF THE DRAWINGS

The invention may be understood upon reading of the following detailed description of non-limiting exemplary embodiments thereof, with reference to the following drawings, in which:

FIG. 1 is a flowchart for describing a method in accordance with some embodiments of the present invention to protect a wearer from inhaling hazardous atmospheres;

FIG. 2 illustrates schematically a respirator device in accordance with some embodiments of the present invention powered by batteries where the respirator device designed to protect the wearer from infecting virus diseases;

FIG. 3 illustrate schematically a respirator device in accordance with some embodiments of the present invention powered by USB connection where the respirator device designed to protect the wearer from infecting virus diseases;

FIG. 4 illustrate schematically a respirator device in accordance with some embodiments of the present invention where the respirator device includes thermoelectric unit;

FIG. 5 illustrate schematically a device in accordance with some embodiments of the present invention where the air that is delivered to the respirator device comes from a fan;

FIG. 6 illustrate schematically a device in accordance with some embodiments of the present invention where the air that is delivered to the respirator device comes from a fan and air filter;

FIG. 7 illustrate schematically a respirator device in accordance with some embodiments of the present invention where the respirator device includes a controller;

FIG. 8 illustrate schematically a respirator device in accordance with some embodiments of the present invention where the insulated chamber includes a Porous heater.

FIG. 9 illustrate schematically a respirator device in accordance with some embodiments of the present invention where the air inlet includes a filter and the heat exchanger element is coated with copper or nano colloid material;

FIG. 10 illustrate schematically a respirator device in accordance with some embodiments of the present invention where the device uses simultaneous application of heating and UV;

FIG. 11 illustrate schematically a desktop device in accordance with some embodiments of the present invention where the device supplies air disinfected a user placed in close vicinity without direct contact with the device;

FIG. 12 illustrate schematically the internal components of the desktop device in accordance with some embodiments of the present invention; and

FIG. 13 illustrate schematically the internal components of the desktop device including TEM in accordance with some embodiments of the present invention.

The following detailed description of the invention refers to the accompanying drawings referred to above. Dimensions of components and features shown in the figures are chosen for convenience or clarity of presentation and are not necessarily shown to scale. Wherever possible, the same reference numbers will be used throughout the drawings and the following description to refer to the same and like parts.

### DETAILED DESCRIPTION OF EMBODIMENTS OF THE INVENTION

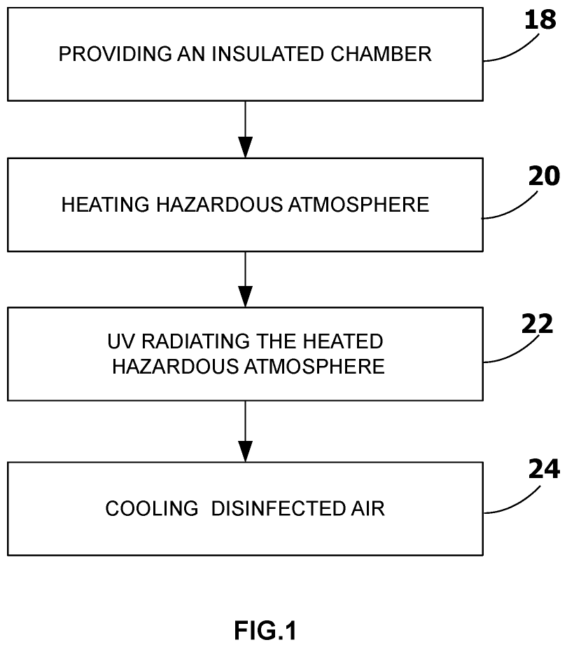

The device and method of the present invention can be configured as a wearer mask or can be configured to be integrated in a vehicle in helmets or in a building/home air conditioning system, or as a desktop device. Referring to FIG. 1, in accordance with the present invention there is provided a method to protect the user from inhaling hazardous atmosphere having virus diseases such as but not limited to coronavirus disease (COVID-19). The method includes the following steps: in step 18 an insulated chamber is provided to insulate the hazardous atmosphere from the user. The insulated chamber may have inlet and outlet and a one-way air valve. In step 20 the hazardous atmosphere is heated and in step 22 the heated hazardous atmosphere is radiated by a UV radiating means in the insulated chamber. In some embodiments steps 20 and 22 can be applied simultaneously by using for example an Intense Pulsed Light (IPL) lamp that both heat and radiates UV radiation. It is well known that UV lamps should not be used on human skin because the UV radiation can cause skin irritation. Thus, steps 20 and 22 are applied in the provided insulated chamber in order that the user will not be exposed to the hazardous atmosphere and the UV radiation. After steps 20 and 22 are processed the air that flows through the insulated chamber outlet is disinfected. However, the disinfected air may be hot after the heating process, thus, before the disinfected air travels to the user's breathing system, the disinfected air is cooled in step 24 by a suitable cooling means which will be detailed in the following specification.

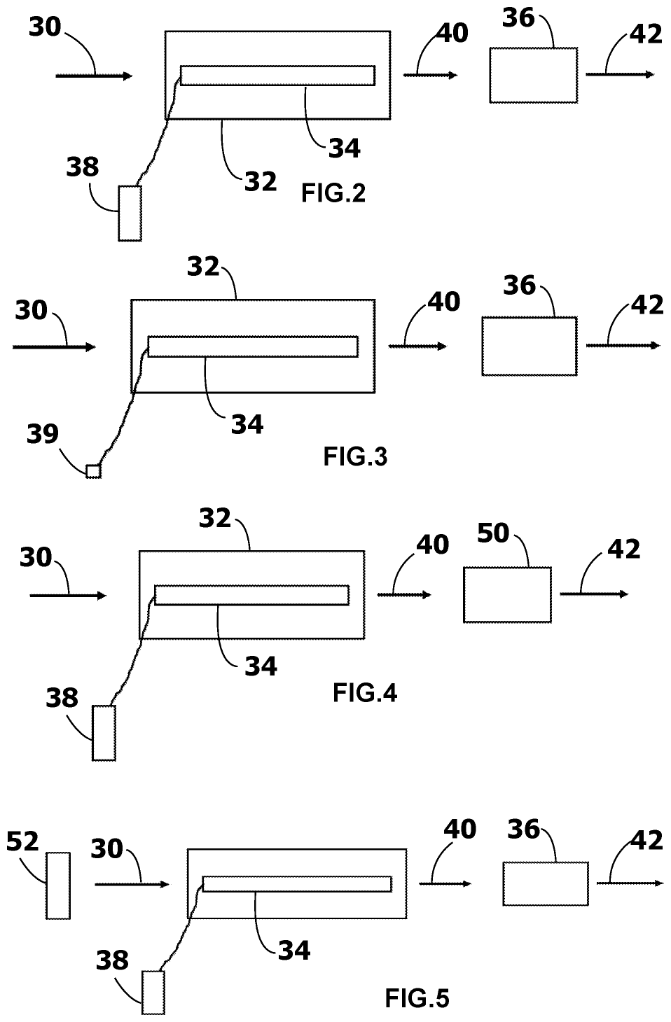

Referring to FIG. 2 there is shown an inlet of hazardous atmosphere or air designated by arrow 30. The respirator device of the invention includes an insulated chamber 32 having a UV radiating and heating means such as but not limited to Pulsed Light (IPL) lamp 34. The air is heated preferably to a temperature of 60 to 100 degrees Celsius however; the heating means can be configured to any desired temperature in order to kill the virus in the air. Insulated chamber 32 has an inlet and outlet. The device of the invention further includes a heat exchanger 36. In operation, the hazardous atmosphere or air 30 travels to the main insulated chamber inlet. The hazardous atmosphere is disinfected by the IPL lamp UVC LED operation 34. The UV and heating means can be powered by any suitable electrical or solar energy means. In this example heating and UV radiating means is powered by a battery 38.

Referring also to FIG. 3 the heating and UV radiating means 34 is powered by using a USB connector 39. After the hazardous atmosphere or air is disinfected in the insulated chamber 32 the heated disinfected air 40 is delivered to the heat exchanger 36 for cooling the disinfected air before the cooled disinfected air 42 is traveled to the user breathing system. The insulated chamber may include one-way air valve for preventing the disinfected air that goes from the outlet of the insulated chamber to travel back to the chamber 32. The one-way air valve can be placed separately from the insulated chamber 32 and designed for enabling the user to exhale more comfortably.

Referring to FIG. 4 in another embodiment of the present invention the unit that is used for heating of hazardous air and cooling the heated disinfected air is a thermoelectric module (TEM) 50 also called a thermoelectric cooler or a Peltier cooler, a semiconductor-based electronic component that functions as a small heat pump, moving heat from one side of the device to the other. By applying a low voltage DC power source to a TEM, heat will be moved through the module from one side to the other. One side of the module face, therefore, will be cooled while the opposite side is simultaneously heated. Consequently, a thermoelectric module may be used for both heating and cooling.

Referring to FIG. 5 in another embodiment of the present invention the device of the invention to protect a user from inhaling hazardous atmospheres can be installed and/or integrated in an air conditional system of a vehicle where the hazardous atmospheres comes from a fan 52 or any means from which the hazardous atmosphere travels from inside the vehicle to the inlet of the insulated chamber 32.

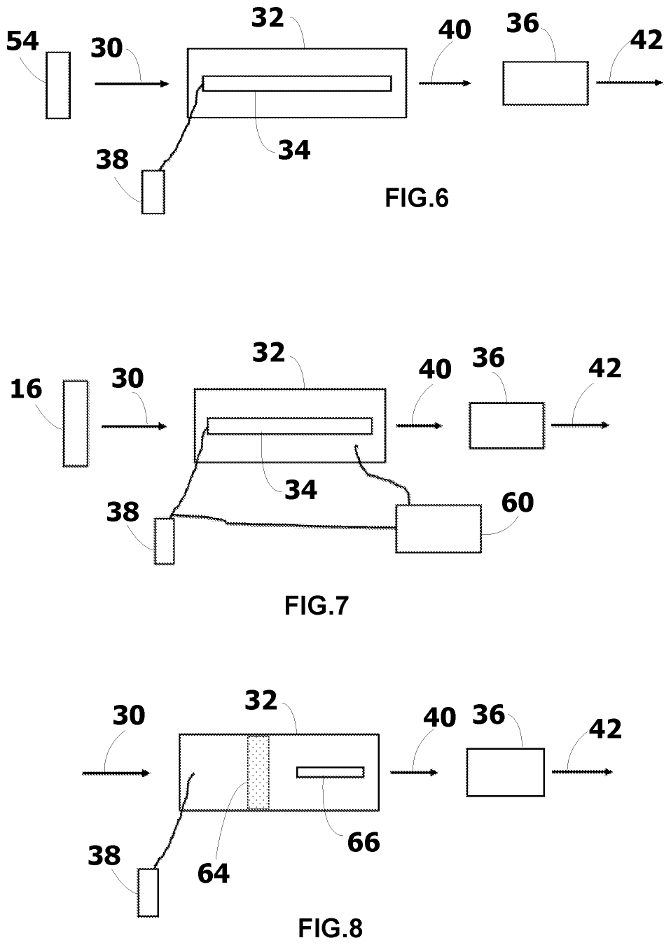

Referring to FIG. 6 in another embodiment of the present invention before the hazardous atmospheres 30 travels to the inlet of the insulated chamber 32 the device of the invention is filtered by a filtering means 54.

Referring to FIG. 7 in another embodiment of the present invention the device of the invention may further include a controller 60 to control the heating temperature of the heating and UV radiating means 34 and cooling means (not shown). The controller 60 may also be able to be used to control the humidity in the insulated chamber 32. The controller 60 may also be able to control the air flow in the insulated chamber 32.

Referring to FIG. 8 in another embodiment of the present invention the insulated chamber 32 includes an air heating unit means such as but not limited to a Porous heater 64 which is a perforated unit through which the air heating process is established in the insulated chamber. the heated air is then being radiated and exposed to UV radiating means 66 for killing any remaining virus. The process of heating and applying UV radiation on the heated air causes the air that travels from the outlet of the insulated chamber 32 to be disinfected. The heating means 64 preferably heats to a temperature of 60 to 100 degrees Celsius.

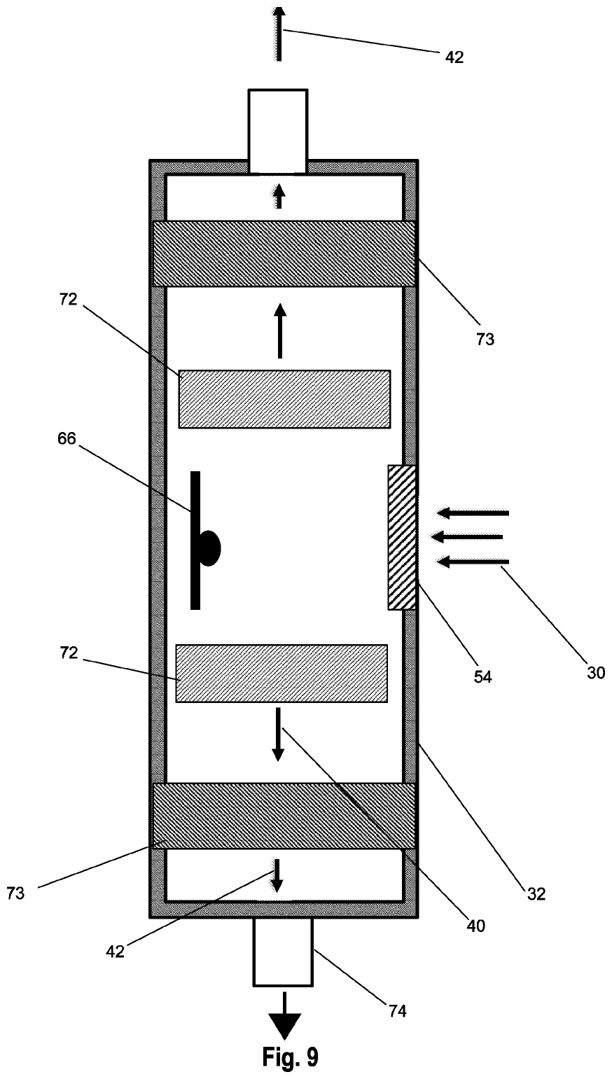

Referring to FIG. 9 there is shown the respirator device of the invention includes a filter 54, insulated chamber 32 having a UV radiating means 66, two heating means 72, two copper plated heat exchanging means 73 and two outlet tubes 74. The air goes through a filter 54 into the insulated chamber 32 where it is simultaneously exposed to UV radiation and heated preferably to a temperature of 60 to 100 degrees Celsius however; the heating means 72 can be configured to any desired temperature in order to kill the virus in the air. Air goes through copper coated heat exchangers 73 where it is cooled down to the desired temperature and then released from the Insulated chamber 32 towards the outlet tube 74 and to the user. The hazardous atmosphere is disinfected by the combination of the steps of heating and exposing the hazardous atmosphere to UV radiation in combination with the use of copper, platinum nano-colloid material or other virus killing material coated on the surface elements such as but not limited to the heat exchanger element 73.

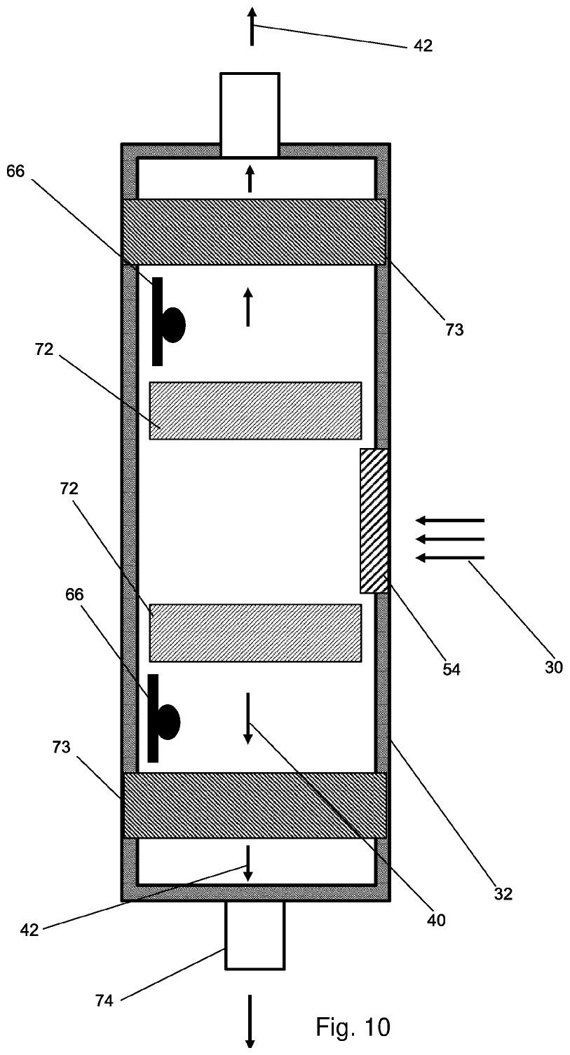

Referring to FIG. 10 the is shown the respirator device of the invention includes a filter 54, insulated chamber 32 having multiple UV radiating means 66, multiple heating means 72, multiple copper plated heat exchanging means 73 and two outlet tubes 74. The air goes through an air filter 54 into the insulated chamber 32 where it is simultaneously exposed to UV radiation and heated preferably to a temperature of 60 to 100 degrees Celsius however; the heating means 72 can be configured to any desired temperature in order to kill the virus in the air. Air goes through copper coated heat exchangers 73 where it is cooled down to the desired temperature and then released from the Insulated chamber 32 towards the outlet tube 74 and the user. The hazardous atmosphere is disinfected by the combination of the steps of heating and exposing the hazardous atmosphere to UV radiation in combination with the use of copper, platinum nano-colloid material or other virus killing material coated on the heat exchanger element 73 causes the hazardous atmosphere to be disinfected by killing any viruses. The use of more than one outlet tube is beneficial for the easing of the respiratory process.

Referring to FIG. 11 illustrate schematically a desktop device 100 for protecting a user 110 from infected air. The device 100 treats air, kills viruses and diffuse clean and disinfected air to the direction surrounding the user's head in such way that the user's head and breathing system receives clean disinfected air. The user 110 inhales air free of viruses. The device 100 can be placed without limitation in homes, workplaces, dining rooms, restaurants and in airplane sits during flights. The device 100 of the present invention having inlet 102 and outlet 104 wherein air (designated by arrow 106) suspected to be infected with a virus enters to device inlet 102. The device 100, positioned for example on a desk 103, supplies disinfected air (designated by arrows 108) from the device outlet 104 to the breathing zone of a user 110 when the user sits or stands in close vicinity with the device 100. One of the benefits of the device 100 is that it does not require wearing a face mask for protecting a user from virus infected air. The protection is provided by providing a disinfected air curtain in the form of constant flow of disinfected air around the user's breathing area. The disinfected air flow is exiting from device outlet 108, to the head of user 110 and thus preventing penetration of infected air to the user breathing zone.

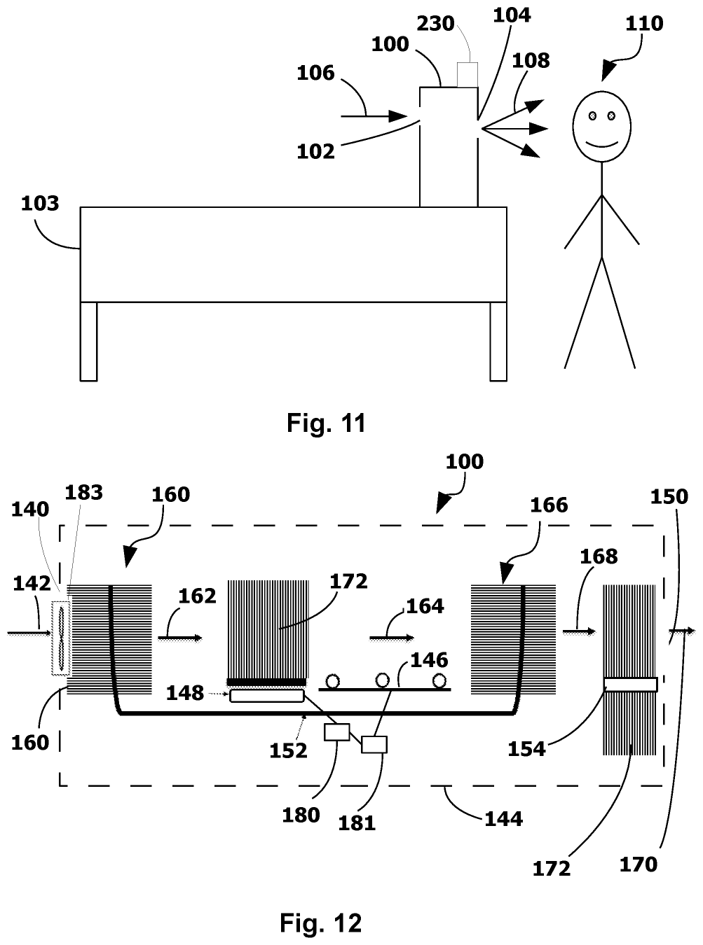

Referring to FIG. 12 there is shown an inlet 140 of hazardous atmosphere or air designated by arrow 142. The disinfecting device 100 in accordance with some embodiments of the present invention includes an insulated chamber 144 having a UV radiating means 146 and heating means 148. The UV radiating means 146 can be for example but not limited to Pulsed Light (IPL) lamp or UV-C LEDs. The air is heated by heating means 148 to a temperature preferably of 60 to 100 degrees Celsius. The heating means 148 can be configured to be any desired temperature in order to kill the virus in the air. Insulated chamber 144 has an inlet 140 and outlet 150. The device 100 further includes a cooling means for example but not limited to combination of Heat Pipe Based Heat Exchanger (HPHE) 152 and ambient heat exchanger 154. HPHE 152 is used for preheating of incoming airflow and precooling of outcoming airflow. The Ambient Heat Exchanger (AHE) 154 is used for cooling of outcoming air flow 170 to temperature close to ambient air temperature.

In operation, the hazardous atmosphere 142 or air travels to the main insulated chamber inlet 140. The hazardous atmosphere is then preheated in the left side fins array 160 of the HPHE 152 then the hazardous atmosphere is heated to the temperature of about 60° C. the air pass through the heater's fins array 160 designating by arrow 162. Air is disinfected by utilizing simultaneous effects of heated air by heating means 148 and UV radiation the air by UV radiating means 146. It is proved that simultaneous application of heat and UV radiation has strong synergy effect and is significantly more efficient than separate application of the mentioned means. After passing preheated air designating by arrow 162 through heater's heat sink 172 with fins plated by copper or colloid the air is heated by heating means 148 and the hot air receives UV treatment designated by arrow 164, the air is precooled by a right section of HPHE 166. The use of HPHE allows the reduction of system power consumption because some of the power applied for air heating is returned to the system and is then used for preheating of the incoming air flow. Precooled air designated by arrow 168 receives additional cooling treatment with the AHE 154 by air passing through the fins array 173 of the AHE 154. Outcoming air flow of cooled clean air is designated by arrow 170 has temperature close to ambient air temperature. All fins of the system elements, Heat Pipe Based Heat Exchanger 166, Heater, Ambient Heat Exchanger 154 can be plated by Copper or Colloid Material providing additional disinfection protection. The UV means 146 and the heating means 148 can be powered by any suitable electrical or solar energy means. In this example heating and UV radiating means is powered by a battery or through a USB connector 180. In another embodiment of the present invention device 100 includes a controller 181 that electrically controls the activation and configures the desired power intensity of the heating means 148 and the UV means 148. The desired temperature of the ambient air 170 can be adjusted by controller 181. Yet in another embodiment of the present invention device 100 may include a fan 183 for more efficiently directing the incoming infected air designated by arrow 142 to left fins array 160 of the HPHE. Yet in another embodiment of the present invention, the device 100 additionally include a tracking system to automatically track the location of the user's 100 head and direct the disinfected air towards the user's breathing area. Yet in another embodiment of the present invention all the mentioned part of device 100 can be replaceable when needed.

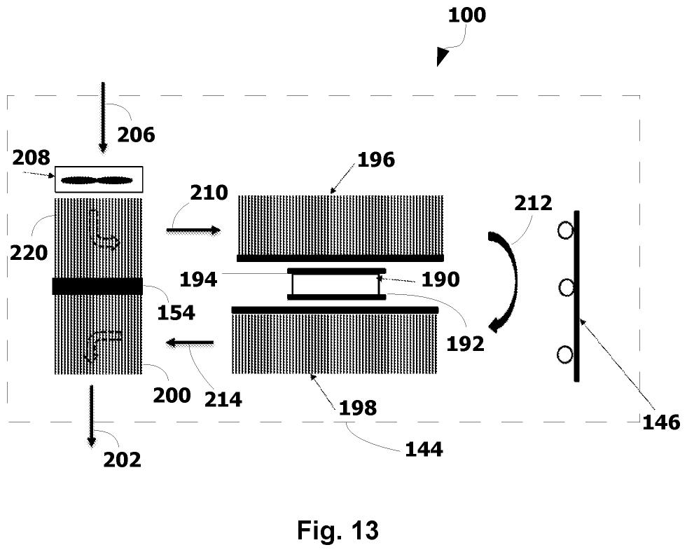

Referring to FIG. 13 in another embodiment of the present invention the unit that is used for heating and cooling of air is a Thermoelectric Module (TEM) 190 also called a thermoelectric cooler or Peltier cooler, is a semiconductor-based electronic component that functions as a small heat pump, moving heat from one side of the device to the other. By applying a low voltage DC power source to a TEM, heat will be moved through the module 190 from one side to the other. One module face 192, therefore, will be cooled while the opposite face 194 simultaneously will be heated. Consequently, a thermoelectric module 190 may be used for both heating and cooling.

The TEM 190 of the invention has fins array 198 and 196 on the both cold and hot side respectively. Heat dissipated on the hot side 196 of TEM fins array 196 is used for heating of air flow to a temperature of 60 to 100 degrees Celsius. Cooling power from the cold side of TEM fins array 198 is used for precooling of disinfected hot air flow after simultaneous heat and UV radiation treatment by the UV radiating means 146. Ambient Heat Exchanger (AHE) 154 in association with the heat exchanger fins array 200 plated by copper or colloid is used for additional cooling and disinfecting of outcoming air flow to the temperature close to ambient air temperature. Arrow 202 designates the cooled cleaned air.

Incoming infected air designated by arrow 206 is drawn by fan 208 and preheated by AHE 154. Preheated air flow designated by arrow 210 passes through the hot side TEM fins array 196 and reaches to temperature, preferably, between 60 to 100 degrees Celsius, after that the hot air passes through UV radiation zone designated by arrow 212 where it is disinfected. The clean air is precooled by TEM cold side fins array 198. Precooled air designated by arrow 214 passes through AHE 154 and reach temperature close to ambient air temperature. Use of AHE 154 allows reducing of the device power consumption by utilizing heat energy of outcoming air for preheating of incoming air flow.

All the fins TEM cold and hot sides array fins 196, 198 and Ambient Heat Exchanger array fins 200 and 220 are plated either by Copper or Colloid Material providing additional disinfection efficiency.

Referring to FIG. 11, in accordance with another embodiment of the present invention device 100 may include face recognition/tracker as known in the prior art for tracking the user face position in respect to the device 100 for efficiently delivering clean air 108 to the user's face by a means that can directs the clean air to the user face which can be any suitable means known in the prior art. Both the face recognition/tracker and the means that directs the clean air to the user face can be controlled by controller 181 that is shown for example in FIG. 12.

It should be understood that the above description is merely exemplary and that there are various embodiments of the present invention that may be devised, mutatis mutandis, and that the features described in the above-described embodiments, and those not described herein, may be used separately or in any suitable combination; and the invention can be devised in accordance with embodiments not necessarily described above.

1. A device to protect a user from inhaling hazardous atmospheres comprising:
an insulated chamber having inlet and outlet;
said chamber includes air heating means and air UV radiating means;
the combination of said air heating means and said UV radiating means is used for disinfecting said hazardous atmospheres;
a cooling means for cooling said disinfected hazardous atmospheres;
wherein, said cooled disinfected atmosphere is then delivered to the user's breathing system.

2. A device according to claim 1 wherein, at least one of the following is coated by a nano-colloid material;
a. said cooling means
b. said heating means.

3. A device according to claim 1 wherein, said UV radiating means is selected from Pulsed Light (IPL) lamp and/or UV-C LEDs.

4. A device according to claim 1 wherein, said air is heated by said heating means to a temperature between 60 to 100 degrees Celsius.

5. A device according to claim 1 wherein, said cooling means is a combination of Heat Pipe Based Heat Exchanger (HPHE) having fins array and ambient heat exchanger (AHE) having fins array, where said HPHE is used for preheating of incoming airflow and precooling of outcoming airflow and said AHE is used for cooling of outcoming air flow to a temperature close to ambient air temperature.

6. A device according to claim 1 wherein said UV means and said heating means can be powered by any suitable electrical or solar energy means; said device may further comprising a controller that electrically controls the activation and configures the desired power intensity of said heating means and said UV means; the desired temperature of said ambient air can be adjusted by said controller that controls said heating means.

7. A device according to claim 5 wherein said device further comprising a fan for more efficiently directing the incoming infected air designated the fins array of said HPHE.

8. A device according to claim 5 wherein said device further comprising a filtering means for filtering the air that enters to the inlet of said device.

9. A device according to claim 1 wherein said device further comprising a tracking system to automatically track the location of said user's head and direct the disinfected air towards the user's breathing area.

10. A device according to claim 1 wherein said heating and cooling means is a thermoelectric Module (TEM).

11. A method to protect a user from inhaling hazardous atmosphere comprising the steps of:
providing insulated chamber having an inlet, outlet; said insulated chamber insulates said hazardous atmosphere from a user;
heating said hazardous atmosphere;
UV radiating on said hazardous atmosphere;
thereby, the hazardous atmosphere is disinfected;
cooling the disinfected atmosphere;
said cooled disinfected atmosphere is then delivered to the user breathing system.

12. A method according to claim 11 wherein, said method further comprising the step of delivering said hazardous atmosphere through fins arrays of Copper or Colloid material for providing additional disinfection protection.

13. A method according to claim 11 wherein, said method further comprising the step of delivering said hazardous air through an air filtering means.

14. A method according to claim 11 wherein, said heating include the use of a Porous heater.

15. A method to protect a user from inhaling hazardous atmosphere wherein, said method is used to disinfect both incoming air and outcoming air, thus further protecting the user's surrounding from an infected user's exhales of infected air by performing at least one of the following steps;
Heating outcoming air in an insulated chamber;
Exposing outcoming air to UV radiation in an insulated chamber;
Exposing outcoming air to nano-colloid materials
Any combination thereof.
